(19) United States
(12) Patent Application Publication
(10) Pub. No.: US 2015/0157240 A1
(43) Pub. Date: Jun. 11, 2015

(54) ELECTRICAL IMPEDANCE IMAGING SYSTEMS

(71) Applicant: General Electric Company, Schenectady, NY (US)

(72) Inventors: David Andrew Shoudy, Niskayuna, NY (US); Jeffrey Michael Ashe, Gloversville, NY (US)

(73) Assignee: General Electric Company, Schenectady, NY (US)

(21) Appl. No.: 14/098,564

(22) Filed: Dec. 6, 2013

(51) Int. Cl. A61B 5/053; A61B 5/00

(52) U.S. Cl. CPC A61B 5/0536; A61B 5/6801; A61B 5/7203

(57) ABSTRACT

An electrical channel having inbuilt current limiting is provided. The electrical channel includes an electrode current source. The electrode current source includes a voltage-to-current converter, a negative impedance converter, and at least one passive current limiting component. Further, the voltage-to-current converter is configured to receive an input voltage and output a corresponding output current. Moreover, the negative impedance converter is operatively coupled to the voltage-to-current converter, where the negative impedance converter is configured to cancel an output impedance of the voltage-to-current converter, a parasitic impedance, or both. Also, the passive current limiting component is configured to limit the output current to a load below a threshold value.

### BACKGROUND

The present specification relates to imaging systems, and more particularly to electrical impedance imaging systems.

Non-invasive monitoring of physiological parameters is desirable to provide useful information for managing a patient's medical condition. For example, shortness of breath and/or difficulty in breathing are directly associated with deteriorating conditions that may result from chronic obstructive pulmonary disease (COPD) or heart failure in patients with heart ailments. Non-invasive monitoring of regional pulmonary ventilation in such patients may help attending caregivers to provide timely medical interventions to manage disease symptoms. Also, the timely management of the disease symptoms may prevent catastrophic events and improve the quality of life of the patient.

Generally, electrical impedance imaging systems, such as, electrical impedance tomography (EIT) or electrical impedance spectroscopy (EIS) systems are used for providing non-invasive monitoring of a patient. Typically, electrical impedance imaging systems include a plurality of electrodes that may be coupled to a subject or an object under investigation. Additionally, a reference electrode may be used in the electrical impedance imaging systems as a reference to a reference potential. Further, the electrical impedance imaging systems may employ a plurality of current sources, where each current source is configured to provide current to a corresponding electrode of the plurality of electrodes. For example, each current source may provide an alternating current to the corresponding electrode. Further, the current source may measure the corresponding voltage appearing at the corresponding electrode. The currents provided to the electrodes may be designed such that a cumulative sum of the currents provided to various electrodes of the plurality of electrodes is zero so that there are no unbalanced currents applied to the subject or object. Based on the applied currents and measured voltages at the electrodes, the electrical impedance imaging systems generate a reconstruction of the conductivity and/or permittivity distribution of the subject or object under investigation.

In an attempt to enhance system performance, some of the electrical impedance imaging systems may apply relatively higher currents or voltages at the electrodes using voltage sources or high output impedance current sources. By applying relatively higher currents or voltages, the applied signal component is increased, enabling enhancements in the signal-to-noise ratio (SNR). However, while it is desirable to increase the applied current or voltage, the current inserted into the patient must be limited over the entire frequency spectrum to preserve patient safety. Limits for acceptable applied currents are defined by standards commissions, such as, but not limited to, the International Electrotechnical Commission (IEC). The values of the current limits typically vary depending on the frequency, such as in the IEC 60601-1 specification. In an electrical impedance spectroscopy system, it is desirable to apply current comprising multiple frequency components to the patient, where the current from each frequency component is near the maximum acceptable current to maximize SNR while maintaining patient safety. However, while attempting to address the concerns regarding patient safety during imaging, some of the existing designs of the electrical impedance imaging systems use current limiting techniques while discounting frequencies of the applied currents. Current limiting while discounting the frequency of the applied currents may unfavorably affect the performance of the electrical impedance imaging systems. For example, current limiting while discounting the frequency of the current may provide sub-optimal results across the frequency band of the applied currents, since a minimum current limit specified by standards commissions, such as the IEC, must be maintained irrespective of the applied frequency, limiting achievable SNR at frequencies where the current limit is higher. There have been attempts to implement the current limiting using software. However, regulatory bodies, such as, but not limited to, the Food and Drug Administration (FDA), prefer implementing electrical safety approaches using hardware for medical devices, such as an electrical impedance imaging system.

### BRIEF DESCRIPTION

Aspects of the present specification relate to an electrical channel having inbuilt current limiting. The electrical channel includes an electrode current source. The electrode current source includes a voltage-to-current converter, a negative impedance converter, a load, and at least one passive current limiting component. Further, the voltage-to-current converter is configured to receive an input voltage and output a corresponding output current. Moreover, the negative impedance converter is operatively coupled to the voltage-to-current converter, where the negative impedance converter is configured to cancel an output impedance of the voltage-to-current converter, a parasitic impedance, or both. Also, the passive current limiting component configured to limit the output current to the load below a threshold value.

In another aspect, a reference current monitor configured to monitor a current at a reference electrode is provided. The reference current monitor includes a reference current-to-voltage converter, a low pass filter operatively coupled to the reference current-to-voltage converter, and a high pass filter operatively coupled to the reference current-to-voltage converter. The reference current monitor further includes a summator operatively coupled to the low and high pass filters.

In yet another aspect, a monitoring and control unit having one or more reference current monitors. The one or more reference current monitors are configured to monitor at least a portion of a reference current appearing at a reference electrode. Further, the one or more reference current monitors are configured to provide respective monitor output signals. Moreover, each of the one or more reference current monitors includes a reference current-to-voltage converter, a low pass filter operatively coupled to the reference current-to-voltage converter, a high pass filter operatively coupled to the reference current-to-voltage converter, and a summator operatively coupled to the low and high pass filters.

In another aspect, an electrical impedance imaging system for imaging a subject is provided. The electrical impedance system includes a plurality of electrodes configured to be disposed on the subject, a reference electrode configured to be disposed on the subject, and a plurality of electrical channels. Further, each electrical channel of the plurality of channels is configured to be operatively coupled to a corresponding electrode of the plurality of electrodes. Additionally, each electrical channel of the plurality of electrical channels includes inbuilt current limiting. Also, each electrical channel of the plurality of electrical channels includes in electrode current source having a voltage-to-current converter configured to receive an input voltage and output a corresponding output current and a negative impedance converter operatively coupled to the voltage-to-current converter. Further, the negative impedance converter is configured to cancel an output impedance of the voltage-to-current converter, a parasitic impedance, or both. Moreover, each electrical channel includes at least one passive current limiting component operatively coupled to the voltage-to-current converter, the negative impedance converter, a load, or combinations thereof, and where the at least one passive current limiting component is configured to limit the output current to the load below a threshold value. The electrical impedance imaging system may also include a processor unit having a physiological parameter extraction module.

### DRAWINGS

These and other features and aspects of embodiments of the present specification will become better understood when the following detailed description is read with reference to the accompanying drawings in which like characters represent like parts throughout the drawings, wherein:

FIG. 1 is a schematic representation of an electrical impedance imaging system having a plurality of electrical channels having inbuilt current limiting, and a monitoring and control unit, in accordance with aspects of the present specification;

FIG. 2 is a block diagram of an example electrical channel having inbuilt current limiting, where the electrical channel includes an enhanced voltage-to-current converter, and an enhanced negative impedance converter, in accordance with aspects of the present specification;

FIG. 3 is a block diagram of an example electrical channel having inbuilt current limiting, where the electrical channel includes two passive current limiting components, in accordance with aspects of the present specification;

FIG. 4 is a block diagram of an example electrical channel having inbuilt current limiting, where the electrical channel includes an enhanced voltage-to-current converter, an enhanced negative impedance converter, and two passive current limiting components operatively coupled to the enhanced voltage-to-current converter, the enhanced negative impedance converter, or both, in accordance with aspects of the present specification;

FIG. 5 is a schematic representation of a circuit topology of an example enhanced Howland circuit, in accordance with aspects of the present specification;

FIG. 6 is a schematic representation of a circuit topology of an example enhanced negative impedance converter, in accordance with aspects of the present specification;

FIGS. 7-10 are example circuit topologies of alternate embodiments of passive current limiting components, in accordance with aspects of the present specification;

FIG. 11 is a graphical representation of simulation results of a maximum output current provided by an electrode current source having an enhanced negative impedance converter, in accordance with aspects of the present specification;

FIG. 12 is a graphical representation of simulation results of a maximum output current provided by an electrode current source having an enhanced negative impedance converter and a passive current limiting component disposed between a Howland circuit and the enhanced negative impedance converter, in accordance with aspects of the present specification;

FIG. 13 is a graphical representation of simulation results of a maximum output current provided by an electrode current source having an enhanced Howland circuit, an enhanced negative impedance converter, a first passive current limiting component disposed between the enhanced Howland circuit and the enhanced negative impedance converter, and a second passive current limiting component, in accordance with aspects of the present specification;

FIG. 14 is a block diagram of a portion of a monitoring and control unit having a reference current monitor configured to monitor a current in a reference electrode, in accordance with aspects of the present specification;

FIG. 15 is a graphical representation of simulation results of an example reference current monitor configured to detect an overcurrent condition in a reference electrode, in accordance with aspects of the present specification;

FIG. 16 is a schematic representation of an example monitoring and control unit configured to detect one or more fault conditions in an electrical impedance imaging system, where the monitoring and control unit includes a single reference electrode, and where the single reference electrode is operatively coupled to two reference current monitors, in accordance with aspects of the present specification; and

FIG. 17 is a schematic representation of an alternate embodiment of a monitoring and control unit, where the monitoring and control unit employs two reference electrodes, and where each reference electrode of the two reference electrodes is operatively coupled to a corresponding reference current monitor, in accordance with aspects of the present specification.

### DETAILED DESCRIPTION

To the extent that the figures illustrate diagrams of the functional blocks of various embodiments, the functional blocks are not necessarily indicative of any division between hardware circuitry. Thus, for example, one or more of the functional blocks (e.g., a processor unit, a monitoring and control unit, an electrical channel, a reference current monitor, or current limiting components) may be implemented in a single piece of hardware or multiple pieces of hardware. Further, it should be understood that the various embodiments are not limited to the arrangements and instrumentalities shown in the drawings.

As used herein, an element or step recited in the singular and preceeded with the word “a” or “an” should be understood as not excluding plural of said elements or steps, unless such exclusion is explicitly stated. Furthermore, references to “one embodiment” are not intended to be interpreted as excluding the existence of addition al embodiments that also incorporate the recited features. Moreover, unless explicitly stated to the contrary, embodiments “comprising” or “having” an element or a plurality of elements having a particular property may include additional such elements not having that property.

In certain embodiments, an electrical impedance imaging system includes a plurality of electrodes that are each configured to receive a determined amount of currents from a corresponding electrical channel of a plurality of electrical channels. Additionally, the electrical impedance imaging system may include a reference electrode. The plurality of electrodes may be disposed on an object or a subject (e.g., a patient). In one example, the plurality of electrodes may employ 32 electrodes and a reference electrode. In this example, a plurality of electrical channels may be made of 32 electrical channels may be used to provide determined amounts of currents to all the 32 electrodes. In one embodiment, the plurality of electrical channels may be configured to provide the determined amounts of currents to the plurality of electrodes in a simultaneous manner.

Moreover, the resultant voltages that appear at the electrodes in response to the applied currents may be measured. The applied currents and the resultant voltages are used to reconstruct an electrical impedance image. By way of example, data representative of the applied currents and the resultant voltages may be processed using a reconstructive technique. Further, the reconstructive technique may be used to generate two dimensional (2D) images or three dimensional (3D) images of an internal conductivity and/or permittivity of the object or the subject. In one embodiment, the applied currents may have sinusoidal waveforms. Also, in one example, a frequency of the current waveforms may be in a range from about 100 Hz to about 10 MHz. In one example, the electrical channels may be configured to provide alternating currents to the electrodes.

Further, each electrical channel of the plurality of electrical channels includes an electrode current source. Moreover, each electrical channel may be configured to provide inbuilt current limiting. In certain embodiments, inbuilt current limiting may be used to control an amount of output current from an electrical channel such that the output current is within a threshold value for applied currents as defined by standards recommended by regulatory bodies. In certain embodiments, inbuilt current limiting may entail a passive current limiting component in a circuit topology of the electrical channel. In particular, the passive current limiting component may be disposed in the electrode current source of the electrical channel. As will be appreciated, safety levels for currents applied to human subjects are governed by regulations. The regulations and standards set forth by the regulatory bodies define maximum threshold current limits that may be provided to the individual electrodes. The currents applied to the human subjects may include intended currents that are generated for a desirable use, unintended currents, currents generated due to a fault in the imaging system, or combinations thereof. It may be noted that the current due to fault conditions may result in undesirable oscillatory waveforms at any frequency with voltages as large as the power supply rails at any output terminal of an amplifier of the electrode current source. Moreover, the current due to fault conditions may result in direct current voltages as large as the rails at any input terminal or output terminal of any amplifier of the electrode current source. Non-limiting examples of the regulatory bodies may include the United States Food and Drug Administration (FDA). It may be noted that regulatory bodies may include regulatory bodies other than the FDA. Further, non-limiting examples of such standards may include IEC 60601-1 standards. Further, it should be noted that although the present specification is described with respect to IEC 60601-1 standards, other standards defined by the regulatory bodies may also be used to define system parameters, such as, but not limited to, current limits, to ensure patient safety during imaging.

By way of example, according to IEC 60601-1 standards, at a current frequency of about 1 kHz, the safe current limit is below 100 microamperes root mean square (μARMS). Whereas, at a current frequency of about 10 kHz, the safe current limit is below 1 milliamperes root mean square (mARMS). Further, at a current frequency of about 100 kHz, the safe current limit is below 10 mARMS.

Some of the existing electrical impedance imaging systems are designed to limit an amount of current received by an electrode to meet the recommended current limits. However, the preferred approach in these existing systems is to apply a constant current limit to all currents over a frequency spectrum. Hence, the existing systems limit the amount of current irrespective of the frequency of the current. In particular, to meet the standards, the existing systems limit the amount of current based on an allowable current at the lowest, and therefore most restrictive, current limit of the frequency spectrum. It may be noted that limiting the current received by the electrode irrespective of the frequency of the current results in the current being significantly limited for some frequencies. For example, limiting the current to 100 μARMS for all frequencies of the frequency spectrum results in the electrodes receiving significantly reduced currents at higher frequencies than otherwise allowed by the prescribed standards. Hence, limiting the current irrespective of the frequency of the current has a degrading effect on the system performance due to reduced dynamic range of the current and worsening sensitivity of the imaging system.

Various embodiments of the present specification provide electrical impedance imaging systems having inbuilt current limiting. As used herein, the term “current limiting” refers to an approach of providing a threshold value for a current that is delivered to a load (e.g., a subject or an object that is to be imaged) or an electrode to facilitate delivery of a determined amount of current based on a frequency of the current as recommended by determined standards, such as, but not limited to, IEC 60601-1 standards, to ensure patient safety during imaging. In certain embodiments, current limiting may be employed in individual electrical channels of the plurality of channels. While employing current limiting in individual electrical channels ensures that current received by each electrode is within a threshold value, however, to ensure that a cumulative current at the reference electrode, which is a sum of all applied currents, is also within a threshold value, referred to herein as “reference threshold value”, the cumulative current at the reference electrode may be monitored. As used herein, the cumulative current at the reference electrode may be referred to as a “reference current”. In same or other embodiments, monitoring and control may be provided at the system-level to ensure that the cumulative current appearing on the reference electrode is below the reference threshold value of the cumulative current.

In certain embodiments, the electrical impedance imaging systems of the present specification may include a plurality of high output impedance electrode current sources that have inbuilt features configured to provide current below the threshold value to an individual electrode. Further, the electrical impedance imaging systems of the present specification may be configured to monitor the cumulative current at the reference electrode. In particular, the electrical impedance imaging systems of the present specification may monitor the cumulative current at the reference electrode to ensure it is less than the reference threshold current. Further, in a non-limiting example, if the cumulative current at the reference electrode is greater than the reference threshold current, the electrical impedance imaging system may be configured to automatically operatively disconnect the plurality of electrode current sources from the electrodes. Advantageously, integrating current limiting into a circuit topology of the one or more electrical channels provides current limiting control below the threshold value without compromising the system performance. Further, the inbuilt current limiting enables the electrical channels to output currents in accordance with the threshold values and frequency profiles defined by the standards and provided by regulatory bodies to provide patient safety and improved performance over the frequency spectrum.

In certain embodiments, the electrical impedance imaging system of the present specification may include an electrode current source that is configured to produce an output current having a frequency in a range from about 100 Hz to about 10 MHz with output impedance of above 10 MOhms It should be noted that higher levels of the output impedance are desirable to enhance a level of precision for electrical impedance imaging for a determined value of applied currents. For example, in healthcare applications, it is highly desirable to obtain or acquire high quality images for purposes, such as, but not limited to, patient monitoring and diagnosis.

In certain embodiments, the electrical impedance imaging systems having current limiting may be configured for extracting/separating or distinguishing electrical measurements of interest from electrical measurements that are not of interest. Non-limiting examples of the electrical measurements of interest may include physiological signals of interest. Non-limiting examples of the electrical measurements that are not of interest may include electrical impedance signals of physiological or non-physiological signals and noise sources that are not of interest. For example, in some embodiments, electrical impedance signals representative of desired physiological activities (e.g., breathing) may be separated from electrical impedance signals representative of undesired physiological activities (e.g., heart or ambulatory motion) and from undesired non-physiological signals (e.g., noise) to perform real-time continuous monitoring of physiological activities. In some embodiments, the electrical impedance imaging systems having inbuilt current limiting may be used for real-time continuous monitoring of physiological activities that may be performed using low complexity electronics and signal processing. In one example, the electrical impedance imaging may be provided in accordance with various embodiments to determine a respiration or breathing rate in patients, such as, but not limited to, comatose, sedated, sleeping, or conscious patients.

It should be noted that although described primarily with respect to monitoring a current in a human subject, the electrical impedance imaging may be used in other applications, such as, but not limited to, defect detection, geological imaging, and process monitoring. Further, it should be noted that the electrical impedance imaging system may be an electrical impedance spectroscopy (EIS) system, or an electrical impedance tomography (EIT) system. Additionally, the integrated current limiting in individual electrical channels as well as the monitoring and control of the reference current may be implemented in connection with any system that is capable of measuring electrical impedance of an object (e.g., a portion of a patient).

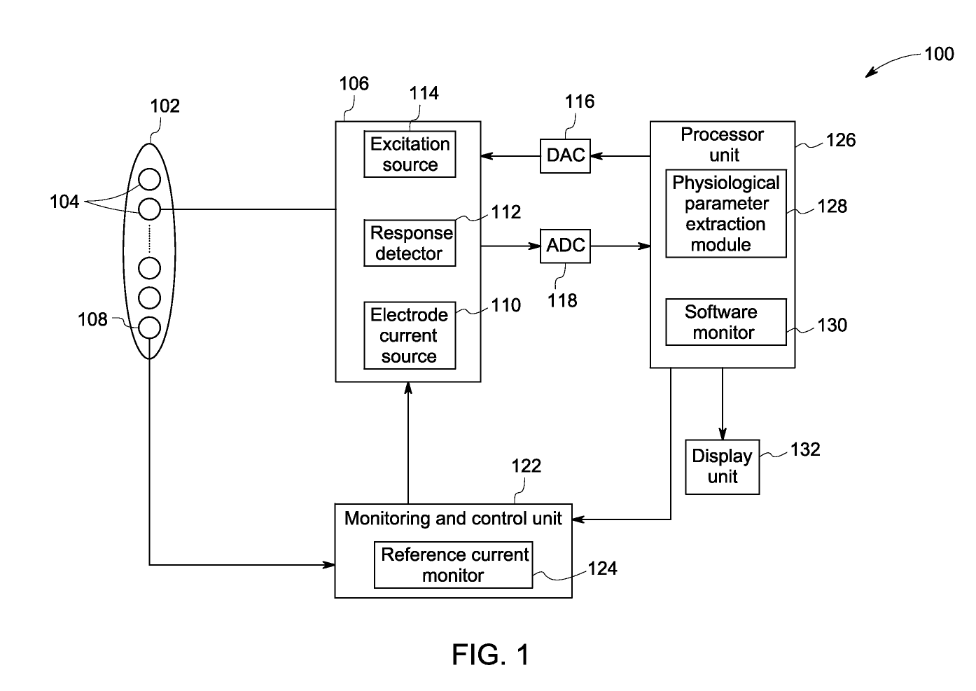

FIG. 1 illustrates an example electrical impedance imaging system 100 in accordance with embodiments of the present specification. The electrical impedance imaging system 100 may be used to obtain electrical impedance measurements of an object 102 (e.g., a patient or subject). The electrical impedance imaging system 100 is an electrode based system. In the illustrated embodiment, the electrical impedance imaging system 100 includes a plurality of electrodes 104 disposed at or proximate a surface of the object 102. By way of example, in a healthcare application (e.g., patient monitoring) the plurality of electrodes 104 may be attached to the skin using a suitable adhesive. The electrodes 104 of the plurality of electrodes 104 may be positioned on the surface of the object 102 in different arrangements and may be driven in different configurations. In one embodiment, the electrodes 104 may be positioned to provide different views of trajectories or angles. In one example, electrodes 104 may be positioned to provide different views of trajectories or angles through lungs, torso, or both. In one example, the views of different trajectories or angles may be used to provide increased sensitivity to breathing and decreased sensitivity to ambulatory motion.

In certain embodiments, the electrodes 104 may be formed from any suitable conductive material used to establish a desirable excitation. For example, the electrodes 104 may be formed from one or more metals such as copper, gold, platinum, steel, silver, and alloys thereof. Other suitable materials for the electrodes 104 may include non-metals that are electrically conductive, such as a silicon based materials used in combination with micro-circuits. In one embodiment, where the object 102 is a human body region, the electrodes 104 may be formed from silver-silver chloride. Additionally, the electrodes 104 may be formed in different shapes and/or sizes, for example, as rod-shaped, flat plate-shaped, or needle-shaped structures.

In operation, the electrodes 104 may be used to deliver electrical current to the object 102 in a continuous or modulated manner such that excitations may be applied across a temporal frequency range (e.g., 100 Hz to 10 MHz) to the surface of the object 102 to generate an electromagnetic field within the object 102. The resulting surface potentials, also referred to as voltages (real, imaginary or complex) on the electrodes 104 may be measured to determine an electrical impedance (e.g. electrical conductivity or permittivity distribution), which is used to separate or distinguish different physiological parameters. Further, in some embodiments, currents driving one or more electrodes 104 may be at different frequencies. In other embodiments, the currents driving the one or more electrodes 104 may be at the same or substantially similar frequency. In some of these embodiments, the currents having the same or substantially similar frequency may have different phase (e.g., 0 degrees, 90 degrees, 180 degrees and 270 degrees). It should be noted that some of the electrodes 104 may have no current applied thereto; such electrodes 104 may be used only for voltage measurements.

Further, in the illustrated embodiment, a reference electrode 108 is one such electrode to which no current is applied. The reference electrode 108 is configured to receive currents from all the electrodes 104. A reference current at the reference electrode 108 is a cumulative sum of the currents applied to the various electrodes 104. Also, the reference electrode 108 is attached to the object to provide a reference potential and is not intended to source or sink the current during normal operation. In one example, the plurality of electrodes 104 may have 32 electrodes 104 to which currents are applied. Further, the system 100 may include a 33^{rd} electrode that may be configured to act as a reference electrode. Although not illustrated, in some embodiments, two or more reference electrodes 108 may be employed in the electrical impedance imaging system 100.

Moreover, in certain embodiments, the electrodes 104 may be operatively coupled to a plurality of electrical channels 106. Although the illustrated embodiment shows only one electrical channel 106, it may be noted that the electrical impedance imaging system 100 may employ a plurality of electrical channels 106. Further, each electrical channel 106 may include an excitation source 114, a response detector 112 and an electrode current source 110.

Further, in some embodiments, each channel 106 of the plurality of electrical channels 106 may have an inbuilt current limiting and may be configured to drive a determined amount of current in the respective electrode 104. A circuit topology of each channel 106 may be same or different from the circuit topology of the other electrical channels 106 of the plurality of electrical channels 106. In one embodiment, each electrode 104 of the plurality of electrodes 104 may be operatively coupled to a corresponding electrical channel 106 of the plurality of electrical channels 106. Each electrical channel 106 of the plurality of electrical channels 106 may be configured to provide a desirable amount of current to the corresponding electrode 104 of the plurality of electrodes 104. Further, the desirable amount of current may be below the threshold value of the current. Further, the electrical channels 106 may be configured to be operatively disconnected from the plurality of electrodes 104. For example, in the case of the cumulative current exceeding the reference threshold value, each electrical channel 106 may be disconnected from its respective electrode 104, thereby avoiding the current with relatively higher values from reaching the electrode 104. As described hereinabove, the current through each electrode 104 as well as the sum of the currents in all the electrodes 104 must be less than the threshold value of the current specified in standards such as the IEC 60601-1 standard defining the specification for medical electrical equipment.

In some embodiments, each electrical channel 106 may employ inbuilt current limiting. The current limiting may be used to control an amount of current travelling from a particular channel 106 to the corresponding electrode 104. The current limiting may be provided to ensure that current values in each of the plurality of channels 106 are below the threshold value. Further, each electrical channel 106 may employ an integrated passive current limiting component, integrated passive stability component, or both. In one non-limiting example, the integrated passive current limiting component, integrated passive stability component, or both may include one or more frequency-sensitive components.

In some embodiments, a topology of the electrode current source 110 may include active elements. In these embodiments, it is desirable to provide current limiting through one or more passive elements. Further, the inbuilt current limiting may be provided using passive hardware elements. In one embodiment, inbuilt current limiting may be provided in a circuit topology of the electrode current source using passive hardware elements, and without using software components or active control loops. Accordingly, in this embodiment, the passive current limiting may be integrated with the electrical channel 106 or the electrode current source 110. Although not illustrated, in some embodiments, the electrode current source 110 may form a part of the excitation source 114. In some of these embodiments, the electrode current source 110 may exist as a separate physical entity within the excitation source 114. Whereas, in some other embodiments, components of the electrode current source 110, such as the voltage-to-current converter, or negative impedance converter, may be integrated with a circuit topology of the excitation source 114. In one embodiment, the electrode current source is a part of the excitation source.

Further, in some embodiments, passive current limiting may be realized in the electrical channels 106 by employing one or more passive current limiting components (not shown). The passive current limiting components may include a circuit topology that is configured to limit an amount of an output current from the electrode current source 110 of the electrical channel 106. The circuit topology of the passive current limiting component may include passive elements, such as, but not limited to, one or more of a resistor, capacitor, inductor or combinations thereof. Moreover, the passive current limiting components may be configured to limit the output current to the electrode 104 below a threshold value. Disadvantageously, using an active current limiting circuit, a fault condition within the active current limiting circuit may in fact compromise patient safety, where the fault condition may include short circuits or oscillations of input or output terminals with voltages as large as the power supply rails of the electrical channels 106. Advantageously, the use of passive current limiting components prevents such undesirable situations in the system 100. Although not illustrated, in some embodiments, the system 100 may employ voltage sources for the electrodes 104 in lieu of some or all of the electrode current sources 106.

Additionally, each electrical channel 106 may include an electrically controlled switch (not shown) configured to be selectively switched on or off to connect and disconnect the corresponding channel 106 from the respective electrode 104. In one example, in operation, the electrically controlled switch may be switched on to facilitate a flow of current from the electrical channel 106 to the electrode 104. However, in instances where the current value increases beyond the threshold value in the electrical channel 106 or at a reference electrode 108, the electrically controlled switch may be configured to switch off, thereby electrically disconnecting the electrical channel 106 and the electrode 104. In one example, the plurality of channels 106 may be disconnected from their respective electrodes 104 using an electronically controlled such, such as, but not limited to, an output relay, configured to electrically disconnect the corresponding channel 106 when an output current to the load (e.g., a patient) is higher than the threshold value.

Further, in addition to the electrode current source 110, each electrical channel 106 may include the excitation driver or excitation source 114 and the response detector 112 that are coupled to the electrodes 104. Also, the excitation source 114 and the response detector 112 are each connected to a processor unit 126 (e.g., a computing device). In one embodiment, the excitation source 114 and the response detector 112 are physically separate devices. In other embodiments, excitation source 114 and the response detector 112 are physically integrated as one element. The processor unit 126 may transmit instructions to the excitation source 114 through a digital to analog converter (DAC) element 116 and receives data from the response detector 112 through an analog to digital converter (ADC) element 118. It should be noted that one or more excitation sources 114 may be provided such that one excitation source 114 is provided per electrode 104, for a subset of electrodes 104 or for all the plurality of electrodes 104.

In various embodiments, a multi-wire measurement configuration is provided that uses different electrodes 104 for excitation from the excitation source 114 and measurement by the response detector 112. Further, in one embodiment, two or more electrical channels 106 may share the same excitation source 114 and/or the response detector 112. In various embodiments, the excitation source 114 applies an excitation current to one or more of the electrodes 104 with a voltage response measured by one or more electrodes 104 of the plurality of electrodes 104. Further, although in the illustrated embodiment a single electrical channel 106 is operatively coupled to an electrode 104, however, it may be noted that each channel 106 of the plurality of channels 106 may be operatively coupled to a corresponding electrode 104 of the plurality of electrodes 104.

Also, in some embodiments, the system 100 may include a monitoring and control unit 122. The monitoring and control unit 122 may include a reference current monitor 124. Further, in one embodiment, the electrically controlled switch of the electrical channel 106 may be activated by the monitoring and control unit 122. While inbuilt current limiting employed in the electrical channels 106 provides current limiting for individual electrical channels 106, the monitoring and control unit 122 is configured to monitor and control the current at the reference electrode 108 below the reference threshold value, thereby providing system-level current limiting. It may be noted that the cumulative current applied to a patient is limited by monitoring the amount of current sunk through the reference electrode 108. Further, in some embodiments, the reference current monitor 124 employed by the monitoring and control unit 122 is configured to monitor the current passing through the reference electrode 108. Further, the reference current monitor 124 may be configured to process the reference current. By way of example, the reference current at the reference electrode 108 may be converted to a voltage using the reference current monitor. Further, the voltage may be amplified and filtered to provide a processed reference voltage. Additionally, the reference current may be compared with the reference threshold value to determine whether the reference current is within the reference threshold value. In instances where the current in the reference electrode 108 is higher than the reference threshold value, one or more hardware components or modules may be used to automatically recognize the overcurrent condition and discontinue the flow of current to the patient, for example, by electrically disconnecting all patient connections in the system.

In certain embodiments, the processor unit 126 includes a software monitor 130 to detect a software related fault in the system 100. Further, the processor unit may include a physiological parameter extraction module 128 within the processor unit 126. Further, the physiological parameter extraction module 128 may be implemented within the hardware or a combination of the software and hardware.

It should be noted that the electrode current source 110 with inbuilt current limiting as well as the reference current monitor 124 and monitor and control unit 122 are performed through hardware components. Further, the individual current limiting and the current limiting at the system-level (at the reference electrode) may be realized using suitable hardware-based circuit topologies, and without any software intervention. It may be noted that hardware-based safety approaches are preferred to software-based approaches by the regulatory bodies.

The system 100 may further include a display unit 132 configured to display the data processed by the processor unit 126. The display unit 132 may include one or more monitors that display patient information, such as including diagnostic images for review, diagnosis, analysis, and treatment. The display unit 132 may automatically display data stored in the memory (not shown) or currently being acquired, this stored data may also be displayed with a graphical representation.

It should be noted that the various embodiments of the present specification may be implemented, for example, in connection with different types of soft-field tomography systems, such as the EIS or EIT, and related modalities.

As discussed hereinabove, each of the electrical channels 106 provides a current to a corresponding electrode 104 via an electrode current source 110. Further, each channel measures the corresponding voltage appearing on the electrode 104. In some embodiments, the applied currents to the electrodes 104 are designed such that the cumulative sum of the currents applied to the plurality of electrodes 104 is zero to avoid any unbalanced currents being applied to the object 102. It should be noted that while in some embodiments, it may be desirable to provide currents such that the cumulative sum of the applied currents is zero so that there are no unbalanced currents at the reference electrode, however, in certain other embodiments, it may be desirable to provide currents such that the cumulative sum of the applied currents is non-zero.

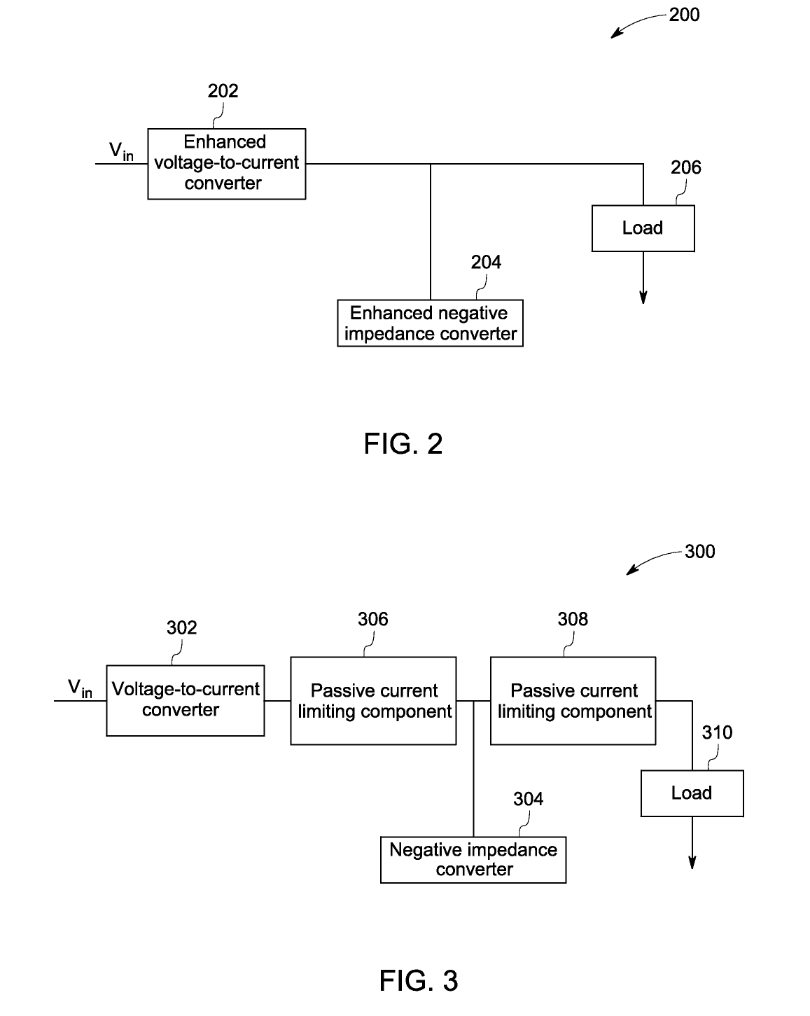

FIG. 2 illustrates a portion of an electrical channel 200 having inbuilt current limiting. The electrical channel 200 may be configured to provide current limiting along with high output impedance. The electrical channel 200 includes an enhanced voltage-to-current converter 202 and an enhanced negative impedance converter 204. In some embodiments, the enhanced negative impedance converter is configured to cancel an output impedance of the enhanced voltage-to-current converter. Further, the enhanced negative impedance converter may be configured to cancel parasitic impedances, stray impedances, or both, in a circuit path to the electrode, thereby facilitating the realization of high output impedance from the electrode current source. As used herein, the term “enhanced converter” refers to a converter having a passive current limiting component disposed in a circuit topology of the converter. Further, the passive current limiting components disposed in the circuit topology of the converter may be referred to as an “integrated passive current limiting component”. By way of example, the voltage-to-current converter 202 may have an integrated passive current limiting component disposed in a circuit topology of a normal voltage-to-current converter to produce the enhanced voltage-to-current converter 202. The enhanced voltage-to-current converter 202 may be configured to receive an input voltage, Vᵢₙ. Further, the enhanced voltage-to-current converter 202 is configured to output a corresponding output current. The enhanced voltage-to-current converter 202 has integrated passive current limiting. In some embodiments, the enhanced voltage-to-current converter 202 may be a Howland circuit. In some of these embodiments, the integrated passive current limiting may be realized in the enhanced voltage-to-current converter 202 by modifying a passive component topology of the Howland circuit design. In addition to the enhanced voltage-to-current converter 202, the electrical channel 200 includes an enhanced negative impedance converter 204 having inbuilt current limiting. The integrated passive current limiting may be realized in the enhanced negative impedance converter 204 by modifying a passive component topology of the negative impedance converter 204.

It may be noted that although in the illustrated embodiment of FIG. 2, both the enhanced voltage-to-current converter 202 and the enhanced negative impedance converter 204 have integrated passive current limiting, however, in alternate embodiments, only one of the enhanced voltage-to-current converter 202 or the enhanced negative impedance converter 204 may have integrated passive current limiting. The electrical channel 200 is further configured to drive an output current produced by the enhanced voltage-to-current converter 202 and the enhanced negative impedance converter 204 through a load 206 in response to the input voltage, Vᵢₙ.

FIG. 3 illustrates a portion of an electrical channel 300 having inbuilt current limiting along with high output impedance. The electrical channel 300 includes a voltage-to-current converter 302 and a negative impedance converter 304. The voltage-to-current converter 302 is configured to receive an input voltage, Vᵢₙ. The voltage-to-current converter 302 is operatively coupled to a first passive current limiting component 306. Further, the negative impedance converter 304 may be operatively coupled to a second passive current limiting component 308. Further, through the combination of the voltage-to-current converter 302 and the two passive current limiting components 306 and 308, the electrical channel 300 is configured to provide a desirable amount of current transmitted to a load 310. It may be noted that, although in the illustrated embodiment of FIG. 3, the electrical channel 300 includes two passive current limiting components 306 and 308, however, in an alternative embodiment, the electrical channel 300 may include one of the two passive current limiting components 306 and 308.

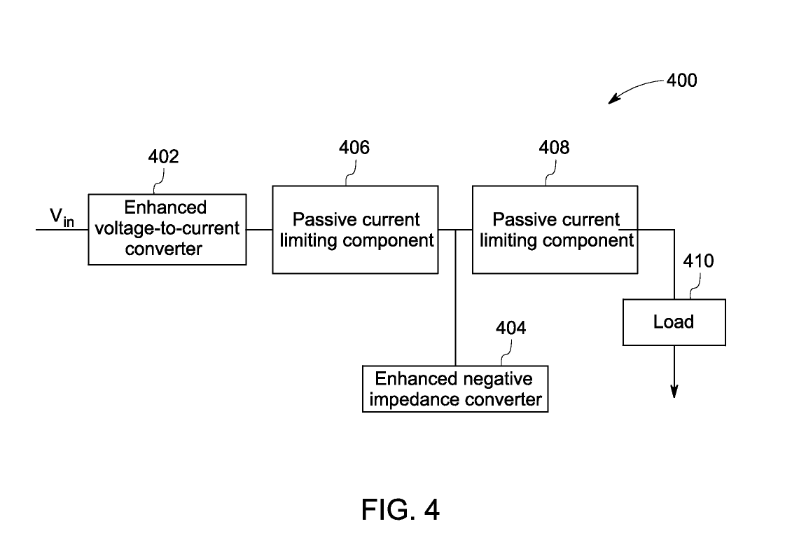

FIG. 4 illustrates a portion of an electrical channel 400 having inbuilt current limiting along with high output impedance. In the illustrated embodiment, the electrical channel 400 includes an enhanced voltage-to-current converter 402 and an enhanced negative impedance converter 404. The enhanced voltage-to-current converter 402 is configured to receive an input voltage waveform generally represented as Vᵢₙ. Further, the enhanced voltage-to-current converter 402 is configured to produce an output current in response to the input voltage waveform, Vᵢₙ received by the enhanced voltage-to-current converter 402. The output current is fed to a load 410. The enhanced voltage-to-current converter 402 may be operatively coupled to a passive current limiting component 406 to provide additional current limiting in the electrical channel 400. Moreover, the enhanced negative impedance converter 404 may be coupled to an output of the passive current limiting component 406 and an input of the load 410. Additionally, the enhanced negative impedance converter 404 may be operatively coupled directly or indirectly to a passive current limiting component 408 to provide current limiting in the electrical channel 400.

Further, the passive current limiting components 406 and 408, and integrated passive current limiting components employed in the enhanced converters 402 and 404 may include passive elements, such as, but not limited to, a resistor, a capacitor, an inductor, or combinations. Additionally, in one embodiment, the integrated current limiting components employed in the enhanced voltage-to-current converter 402 and the enhanced negative impedance converter 404, and the passive current limiting components 406 and 408 may include a passive resistor-capacitor component. In one example, the passive resistor-capacitor component may be frequency-sensitive resistor-capacitor components.

It may be noted that the embodiment illustrated in FIG. 4 is a specific example of the electrical channel 400 that can be employed in an electrical impedance imaging system of the present specification to provide passive current limiting, however, in alternate embodiments, various combinations of the enhanced voltage-to-current converter 402, enhanced negative impedance converter 404, and the passive current limiting components 406 and 408 may be employed in the electrical channel 400. For example, although not illustrated, in an alternate embodiment, one or both of the enhanced voltage-to-current converter 402 or the enhanced negative impedance converter 404 may be replaced with similar converters but without inbuilt current limiting. Alternatively, or in addition, only one of the passive current limiting components 406 and 408 may be employed in the electrical channel 400. In a specific alternate example, the enhanced negative impedance converter 410 may be replaced with a negative impedance converter that does not have an inbuilt current limiting. Further, in this example, only the passive current limiting component 408 may be employed in the electrical channel 400.

In one embodiment, the enhanced voltage-to-current converter 402 may include a Howland circuit. The passive current limiting component 406 disposed between the enhanced voltage-to-current converter 402 and the enhanced negative impedance converter 404 may be used to further limit an amount of current coming from the voltage-to-current converter 402. In addition to providing current limiting, the passive current limiting components 406 and 408 may be used to shape a frequency profile of the output current of the electrical channel 400. The output current having a desirable curve shape may be used to facilitate relatively high current output for a given frequency of current.

In certain embodiments, the electrical channels 200, 300 and 400 (see FIGS. 2, 3, and 4, respectively) may be configured to provide a high output impedance while simultaneously limiting the amount of current transmitted to a corresponding electrode coupled to that electrical channel. Further, the electrical channels 200, 300 and 400 may be configured to maintain high output impedance with varying load. In one embodiment, the individual electrical channels 200, 300 or 400 with inbuilt current limiting may be suitable for use in electrical impedance imaging systems, electrical impedance tomography systems, or both. Further, it may be noted that voltage-to-current converters, negative impedance converters, and passive current limiting components may be employed in an electrode current source of an electrical channel.

It may be noted that the use of the negative impedance converter may reduce or remove the parasitic impedance in a high output impedance current source. The use of the negative impedance converter is associated with instability in the circuit. Hence, in some embodiments, additional circuit stability elements may be introduced. Moreover, depending on the frequency of operation, passive current limiting may be implemented at one or more locations discussed with regards to FIGS. 2-4. Further, the passive current limiting may be employed as integrated passive current limiting, where the current limiting component is integrated with a circuit topology of the voltage-to-converter and/or the negative impedance converter, or by using individual passive current limiting components that are operatively coupled to the voltage-to-converter or negative impedance converter. Each of such modifications in the electrical channel may facilitate intrinsically and passively limiting the output current from the voltage-to-impedance converter and/or the negative impedance converter during intended operation, unintended operation, fault conditions, or combinations thereof. Further, as discussed hereinabove, each of these modifications directed to providing passive current limiting in the electrical channel may be used alone or in conjunction with each other to provide desirable current limiting while maintaining high output impedance. In certain embodiments, the current limiting is performed entirely through hardware circuits with no software intervention. Hardware-based safety approaches are preferred to software-based approaches by regulatory bodies.

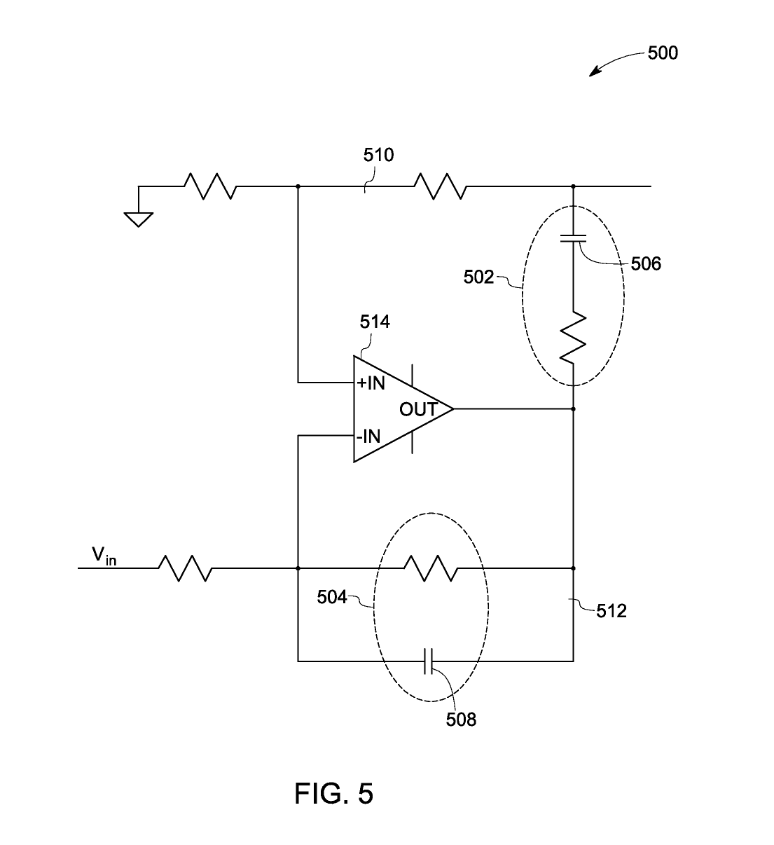

FIG. 5 illustrates an example circuit topology 500 of an enhanced voltage-to-current converter having inbuilt current limiting. In particular, the circuit topology represents an enhanced Howland circuit. The enhanced Howland circuit may be formed by modifying a Howland circuit to include integrated passive current limiting components. Further, the enhanced Howland circuit may be modified to facilitate circuit stability. Additionally, the enhanced Howland circuit may be modified to increase output impedance of the Howland circuit to a desirable extent.

In the illustrated embodiment, the circuit topology 500 of the enhanced Howland circuit includes an integrated passive current limiting component 502 and an integrated passive stability component 504. It should be noted that number and locations of capacitors, such as, the capacitors 506 and 508 of the passive current limiting component 502 and passive stability component 504 may vary depending on the requirement for current limiting and system stability. The enhanced Howland circuit 500 may further include an operational amplifier 514. The capacitor 506 of the passive current limiting component 502 may be disposed in an output feedback loop 510 of the Howland circuit. Further, the capacitor 508 of the passive stability component 504 may be disposed in a gain feedback loop 510 of the Howland circuit. In some embodiments, the passive current limiting component 502 disposed in the output feedback loop 510 facilitates limiting high frequency currents, while the passive stability component 504 disposed in the gain feedback loop is configured to provide circuit stability in the enhanced Howland circuit 500.

Additionally, although in the illustrated embodiment of FIG. 5, the passive current limiting component 502 and the passive stability component 504 are illustrated as resistor-capacitor components, it should be noted that in alternative embodiments, the passive components 502 and 504 may include one or more resistors, capacitors, inductors, or combinations thereof. In particular, the passive components 502 and 504 are specific examples, and multiple possible configurations of resistors, inductors and capacitors may be employed in the passive components 502 and 504. For example, the passive current limiting component 506 and the passive stability component 508 may include a single passive element, such as, but not limited to, a resistor, a capacitor, an inductor, or combinations thereof. Alternatively, the passive components 502 and 504 may include a plurality of one or more of these passive elements. Further, the passive elements may be coupled to one another in series and/or parallel combinations. Moreover, although not illustrated, in one embodiment, one or more capacitors or other passive components may be used in the enhanced Howland circuit topology 500 to facilitate circuit stability.

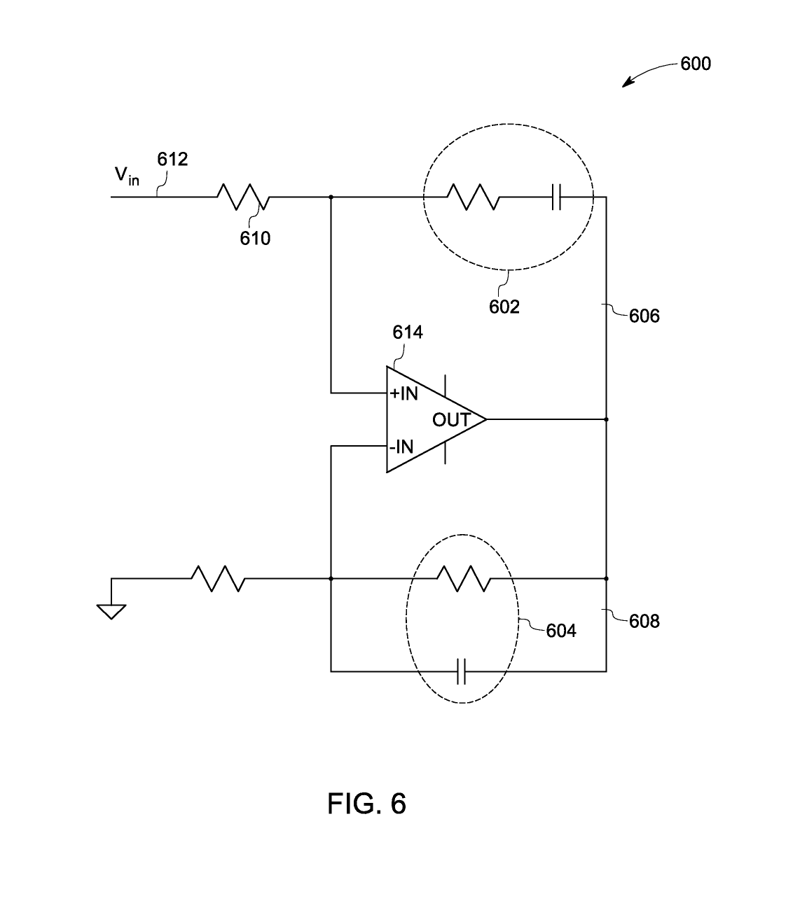

FIG. 6 illustrates an example circuit topology 600 of an enhanced negative impedance converter formed by providing integrated passive current limiting in a circuit topology of a negative impedance converter. The circuit topology 600 of the enhanced negative impedance converter employs an integrated passive current limiting component 602 and an integrated stability component 604. The circuit topology 600 further includes an operational amplifier 614. The integrated passive current limiting component 602 may be disposed in an output feedback loop 606. Further, the integrated stability component 604 may be disposed in a gain feedback loop 608. Additionally, the enhanced negative impedance converter 600 may include a resistor 610 disposed in an input feed branch 612. The integrated passive current limiting component 602 disposed in the output feedback loop 606 in conjunction with the resistor 610 disposed in the input feed branch 612 may be configured to limit high frequency currents in the enhanced negative impedance converter 600.

It should be noted that the integrated passive components 502 and 504 of FIG. 5 and the integrated passive components 602 and 604 of FIG. 6 are merely one possible example of various possibilities of passive components that may be employed in the enhanced Howland circuit 500 (see FIG. 5) or the enhanced negative impedance converter 600 (see FIG. 6). By way of example, different number of resistors may be employed in place of the resistor 610 of FIG. 6. Furthermore, different number of resistors, capacitors, inductors, or combinations thereof may be employed in the passive components 502, 504, 602 and/or 604 to provide current limiting for low frequency current, high frequency current, or both.

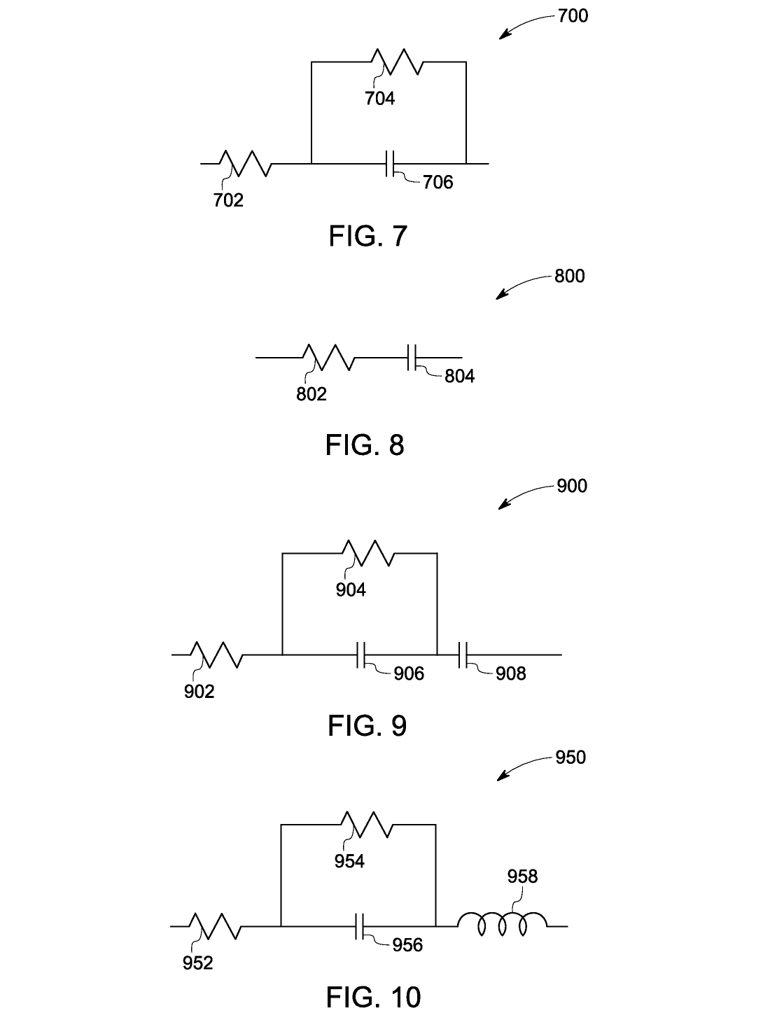

FIGS. 7-10 illustrate alternative embodiments of non-limiting examples of passive current limiting components that may be employed in one or more electrical channels of the present specification. The passive current limiting components may be configured to act as integrated current limiting components, where the circuit topology of the passive current limiting components may be integrated in the circuit topology of a voltage-to-current converter or a negative impedance converter. Further, the passive current limiting component may be configured to be operatively coupled to the voltage-to-current converter or a negative impedance converter of an electrode source to form an electrical channel having inbuilt current limiting. Moreover, it may be noted that the number and locations of resistors, capacitors and inductors may vary in the illustrated embodiments of FIGS. 7-10.

Turning now to FIG. 7, a circuit topology of a passive current limiting component 700 may include resistors 702 and 704. Further, the circuit topology of the passive current limiting component 700 may include a capacitor 706. The resistor 704 and the capacitor 706 are in parallel combination. Further, the parallel combination of the resistor 704 and the capacitor 706 are coupled to the resistor 702 in series. Alternatively, although not illustrated, the passive current limiting component 700 may simply employ a single resistor. In one example, the passive current limiting component 700 may be employed in place of the passive current limiting components 406 and/or 408 of FIG. 4.

FIG. 8 illustrates a circuit topology of a passive current limiting component 800 having a resistor 802 and a capacitor 804. The resistor 802 and the capacitor 804 are coupled in series. In one example, the passive current limiting components 406 and/or 408 of FIG. 4 may be replaced with the passive current limiting component 800 of the presently contemplated embodiment of FIG. 8.

FIG. 9 illustrates a circuit topology of a passive current limiting component 900 employing resistors 902 and 904, and capacitors 906 and 908. The resistor 902 and the capacitor 908 are coupled in series to a parallel combination of the resistor 904 and the capacitor 906. The passive current limiting components 406 and/or 408 of FIG. 4 may be replaced with the passive current limiting component 900 of the presently contemplated embodiment of FIG. 9. It may be noted that the capacitors 804 (see FIGS. 8) and 908 (see FIG. 9) may be configured to facilitate blocking direct current (DC) in a system safety circuit. In particular, the capacitors 804 and 908 may be configured to block direct current from entering the load. Alternately, although not illustrated, the passive current limiting components 800 and 900 may simply employ a single capacitor.

FIG. 10 illustrates a circuit topology of a passive current limiting component 950 employing resistors 952 and 954, a capacitor 956, and an inductor 958. The resistor 952 is coupled in series to a parallel combination of the resistor 954 and the capacitor 956. Further, the inductor 958 is coupled in series to the parallel combination of the resistor 954 and the capacitor 956. In one example, the passive current limiting component 406 and/or 408 of FIG. 4 may be replaced with the passive current limiting component 950 of the presently contemplated embodiment of FIG. 10.

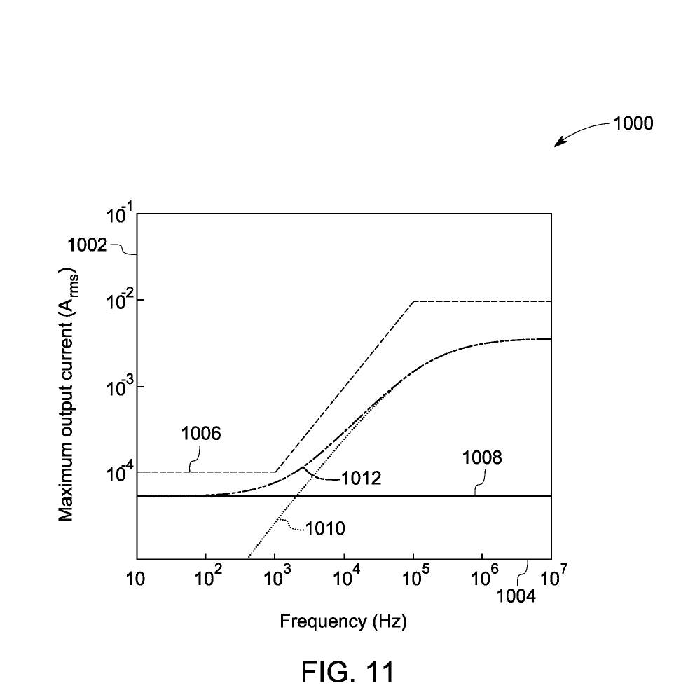

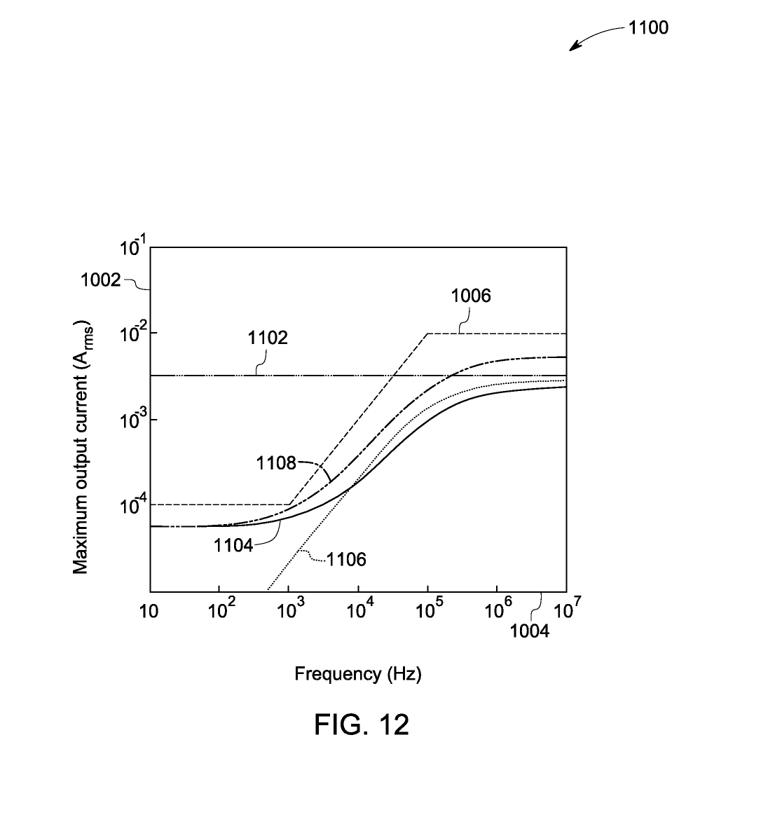

FIGS. 11-13 illustrate maximum possible currents that may enter a load from the electrical channel. The maximum currents represented in FIGS. 12-14 may include desirable commanded currents as well as currents due to fault conditions in the system. The fault conditions may result in oscillatory voltages as large as the rails of the power supplies. Further, the oscillatory voltages may occur at any of the amplifier outputs. Further, the fault conditions may result in direct current voltages as large as the rails at one or more of an input terminal or output terminal of the amplifiers.

FIG. 11 illustrates simulation results 1000 for a maximum output current (ordinate 1002) vs. frequency of an output current (abscissa 1004) provided by an electrode current source of an electrical channel. It is noted that “maximum output current” refers to the maximum possible current that may be applied to the human subjects, which may include intended currents that are generated for a desirable use, unintended currents, currents generated due to a fault in the imaging system, or combinations thereof. A curve 1006 represents current limits defined in accordance with IEC 60601-1 current limits over a range of frequencies as defined by regulatory bodies. In particular, the current limits represented by the curve 1006 represent the maximum possible current that may be passed to a load of an electrode current source. Further, a curve 1008 represents the maximum possible output current from a regular Howland circuit, and a curve 1010 represents the maximum possible output current from an enhanced negative impedance converter. Due to the modification in the enhanced negative impedance converter, the current limiting in the enhanced negative impedance converter maintains the maximum possible output current of the enhanced negative impedance converter within the IEC 60601-1 current limits As a result, the combined maximum circuit current may follow the maximum current limits (i.e., threshold value of the current) over the spectrum of frequencies. Further, the combined maximum current, which is computed as the sum of the maximum current of the Howland circuit, represented by the curve 1008 and the maximum current of the enhanced negative impedance converter, represented by the curve 1010 may be represented by reference numeral 1012. In some embodiments, the shape of the curve 1012 representing the combined maximum current may be such that the current limits defined by the curve 1012 over the frequency spectrum match the IEC 60601-1 current limits. In one example, the curve 1012 representing the combined maximum current may have S-curve shape.

As illustrated in FIG. 11, by only modifying the negative impedance converter, the maximum Howland current 1008 at higher frequencies may be maintained at relatively lower values than allowed by the IEC 60601-1 current limits In general, the current limits at higher frequencies may be maintained at relatively lower values to meet the IEC 60601-1 current limits at lower frequencies. It may be noted that increasing the applied current limits to the patient while being within the threshold value may result in a higher signal to noise ratio (SNR). Accordingly, since the maximum current at the Howland circuit is relatively lower at least for the higher frequencies than maximum allowable limits, the signal to noise ratio for the higher frequencies is below optimal. For example, the maximum applied current at the Howland circuit at frequencies above 1 kHz may be below optimal, hence, the signal to noise ratio at frequencies above 1 kHz may be relatively low compared to the achievable signal to noise ratio when applying currents that are closer to the IEC 60601-1 current limits.

FIG. 12 illustrates simulation results 1100 for an electrode current source employing a Howland circuit and an enhanced negative impedance converter. Further, the electrode current source includes a passive current limiting component disposed between the Howland circuit and the enhanced negative impedance converter to limit the current from the Howland circuit. Further, the passive current limiting component disposed between the Howland circuit and the enhanced negative impedance converter may also be configured to simultaneously shape the frequency profile of the combined current to track the S-curve shape of the IEC 60601-1 limits A curve 1102 represents current by the Howland circuit. A curve 1104 represents current after inclusion of the passive current limiting component between the Howland circuit and the enhanced negative impedance converter. The curve 1104 satisfies the current limits set by IEC 60601-1 limits over the frequency spectrum. A curve 1106 represents the maximum possible output current by the enhanced negative impedance converter.

By adding the passive current limiting component between the Howland circuit and the enhanced negative impedance converter, the Howland gain may be increased. Further, the increase in the Howland gain substantially improves the SNR at relatively higher frequencies in the frequency spectrum. It may be noted that if only a Howland circuit is used, the Howland gain may exceed IEC 60601-1 limits at lower frequencies as shown by the curve 1102, however, with the addition of the passive current limiting component between the Howland circuit and the enhanced negative impedance converter, the current limit, as shown by the curve 1104, follows the IEC 60601-1 limits by maintaining lower gain at low frequencies and higher gain at higher frequencies. In the illustrated example of FIG. 12, the enhanced negative impedance converter may include a modified negative impedance converter to include current limiting within the positive feedback loop.

The combined current from the Howland circuit and the enhanced negative impedance converter with a passive limiting component added between the Howland circuit and enhanced negative impedance converter, represented generally by reference numeral 1108, may follow a S-curve shape that tracks the IEC 60601-1 limits over the frequency spectrum. Advantageously, the S-curve shape of the combined current 1108 does not have the drawback of low SNR. The electrode current source having the current limits represented in FIG. 12 may include one or more passive current limiting components illustrated in FIGS. 7-10.

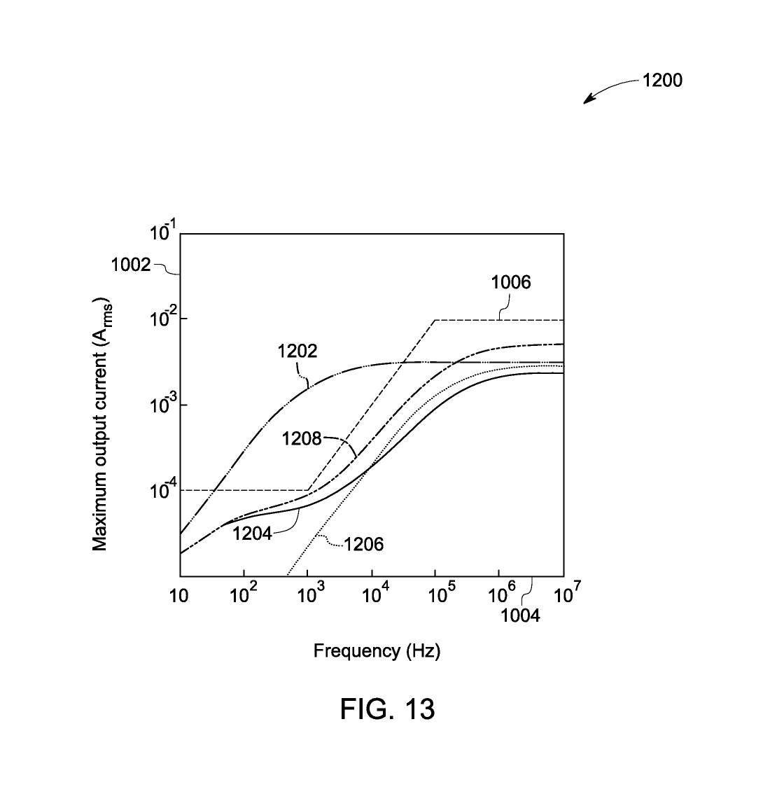

FIG. 13 illustrates current limits for an electrical channel having an arrangement similar to the one illustrated in FIG. 4. Further, FIG. 13 represents an example output from the electrical channel of FIG. 4. In particular, the current limits are for an electrical channel that includes an enhanced Howland circuit and an enhanced negative impedance converter. In one example, the enhanced Howland circuit may include a passive current limiting component. The graphical representation 1200 illustrates current limits 1202 for an enhanced Howland circuit and current limits 1206 for an enhanced negative impedance converter. Further, the curve 1204 represents current limits for the enhanced Howland circuit with the first passive current limiting component disposed between the enhanced Howland and enhanced negative impedance converter. Moreover, curve 1208 represents the sum of curves 1204 and 1206 and is representative of the overall maximum current of the circuit.

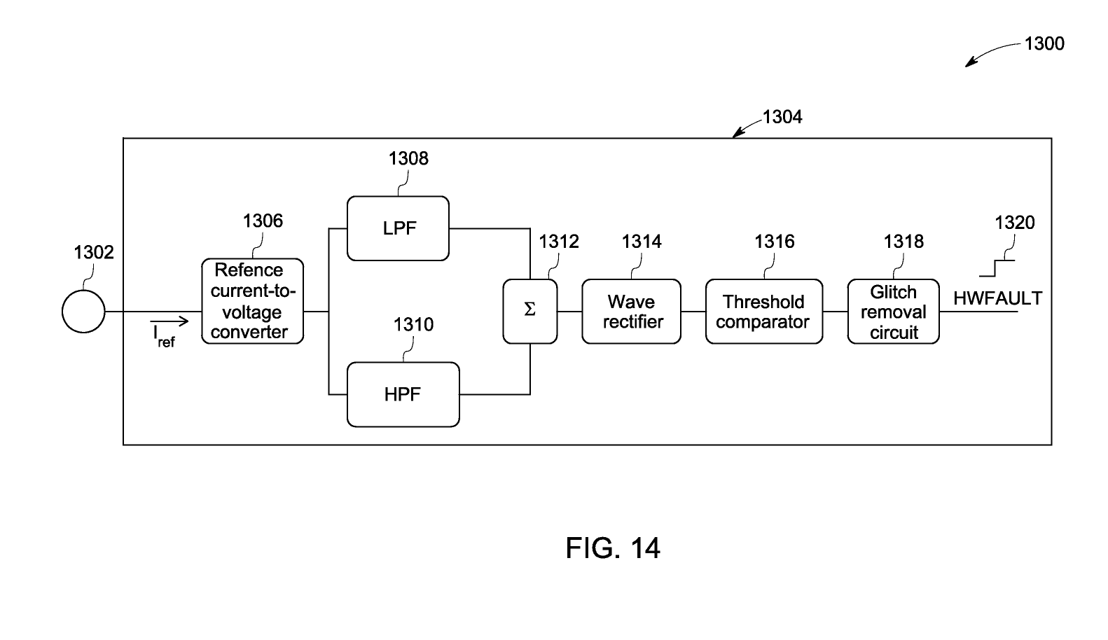

FIG. 14 illustrates a graphical representation of a portion 1300 of an electrical impedance imaging system having a reference current monitor 1304 that is operatively coupled to a reference electrode 1302. In certain embodiments, the reference current monitor 1304 may be used to measure a cumulative current (I_{ref}) at the reference electrode 1302. In some embodiments, a value of the cumulative current at the reference electrode 1302 may be balanced. In these embodiments, a value of the cumulative current may be zero. In some other embodiments, the value of the cumulative current may be unbalanced. Further, in these embodiments, the value of the cumulative current may be non-zero. In one example, the unbalanced cumulative current may result from an intended operation of the system, an unintended operation of the system, fault conditions in the system, or combinations thereof. Further, the unbalanced cumulative current may occur at the operating frequency or at arbitrary frequencies. Furthermore, the unbalanced cumulative current may be synchronized with the system operation or may be asynchronous with the system operation. Moreover, the unbalanced cumulative current may be continuous or may be transient.

The reference current monitor 1304 may be configured to monitor current conditions (e.g., balanced or unbalanced current conditions) in the electrical impedance imaging system. Conventional approaches for monitoring the cumulative current value at the reference electrode use broadband circuits. However, these conventional approaches require digital processing to determine whether an overcurrent condition exists at the reference electrode. In the embodiments disclosed in the present specification, the reference current monitor 1304 may primarily rely on hardware components to monitor the current conditions of the reference electrode 1302. In one example, the reference current monitor 1304 may rely only on hardware components to monitor the current conditions of the reference electrode 1302. Advantageously, the use of the hardware components by the reference current monitor 1304 for monitoring the current conditions may provide intrinsic safety, and abide by the recommendations of the regulatory bodies. In some embodiments, the reference current monitor 1304 may monitor the current conditions at the reference electrode 1302 without using any software components or active control loops.

Further, the reference current monitor 1304 may be configured to provide a reference potential from which any unbalanced cumulative current may be sourced or sunk. It may be noted that a condition of the system where the cumulative current at the reference electrode 1302 is greater than the reference threshold value may be referred to as an “overcurrent fault condition”. The reference threshold value of the unbalanced cumulative current may be based on the current limits defined by the regulatory bodies. In a non-limiting example, if one or more electrodes are not properly coupled to the patient, an unbalanced cumulative current may appear at the reference electrode 1302. In instances where the unbalanced cumulative current exceeds the reference threshold value, an overcurrent fault condition may be identified by the system and suitable actions may be performed by the system to ensure patient safety. For example, upon identification of the overcurrent fault condition, the system may be configured to electrically disconnect the electrodes from their respective electrical channels. In one embodiment, the unbalanced cumulative current may be drained out using hardware structure. In a particular embodiment, the unbalanced cumulative current may be drained out from the reference electrode 1302 using only hardware components without any intervention by software components. It may be noted that in some embodiments it may be desirable to have a non-zero or unbalanced value of the cumulative currents on the reference electrode 1302. While in some other embodiments, it may be desirable to have an unbalanced cumulative current value.

In some embodiments, the reference electrode 1302 may be coupled to a single reference current monitor 1304. In some other embodiments, the reference electrode 1302 may be operatively coupled to two or more reference current monitors 1304. In these embodiments, each of the reference current monitor 1304 of the two or more reference current monitors 1304 may be same or different. Further, in some embodiments, the electrical impedance spectroscopy system of the present specification may employ one or more reference electrodes 1302. Further, the one or more reference electrodes 1302 may be coupled to one or more corresponding reference current monitors 1304. In one example, in instances where the electrical impedance spectroscopy system employs two or more reference electrodes 1302, each reference electrode 1302 of the two or more reference electrodes 1302 may be coupled to a corresponding reference current monitor 1304. In another example, each reference electrode 1302 of the two or more reference electrodes 1302 may be coupled to two or more corresponding reference current monitors 1304. In some embodiments, a reference electrode 1302 may be configured to provide a reference potential. Additionally, the reference electrode 1302 may also be configured to act as a current source or current sink in the event of an unbalanced cumulative current from a plurality of electrode current sources (not shown). In one example, the reference electrode 1302 may be attached to a shoulder area of a patient. However, the location of the reference electrode 1302 may vary depending on the application. By way of example, in the case of monitoring of lungs of the patient, the reference electrode 1302 may be disposed near the shoulder area. In one embodiment, the reference electrode 1302 may be relatively larger in size as compared to other electrodes of the plurality of electrodes. In one example, the reference electrode 1302 may be coupled to the patient using an adhesive. In same or different example, the reference electrode 1302 may be rectangular in shape.

In certain embodiments, the reference electrode 1302 may be configured to act as a virtual reference configured to collect the cumulative current. The cumulative current (I_{ref}) collected at the reference electrode 1302 may be passed through a reference current-to-voltage converter 1306 to provide a voltage signal that is representative of the cumulative current, I_{ref.} Further, the voltage signal may include frequency components of the cumulative current, I_{ref.} The frequency components of the voltage signal may be segregated based on frequencies. By way of example, the frequency components of the voltage signal that are below a determined frequency value may be separated from the frequency components above a determined frequency value by using filters. In one embodiment, a low pass filter 1308 may be used to separate out the frequency components that are below the determined frequency value. Further, a high pass filter 1310 may be used to separate out the frequency components that are above the determined frequency value. In one embodiment, the low frequency components of the voltage signal may have a frequency value that is less than about 1 kHz. In same or different embodiment, the high frequency components of the voltage signal may have a frequency value that is more than about 100 kHz. The voltage signal at the output of the low pass filter 1308 may be thus sensitive to unbalanced cumulative currents having lower frequency values. However, the voltage signal at the output of the low pass filter 1308 may not be as sensitive to unbalanced cumulative currents having higher frequency values. The voltage signal at the output of the high pass filter 1310 may be thus sensitive to unbalanced cumulative currents that have higher frequency values; however, the voltage signal at the output of the high pass filter 1310 may not be sufficiently sensitive to unbalanced cumulative currents having lower frequency values.

The voltage signals at the output of the low and high pass filters 1308 and 1310 may be weighted and combined at a summator 1312 to provide a summed voltage signal that is proportionally sensitive to different frequency components of unbalanced cumulative currents collected at the reference electrode 1302, where the different frequency components may have high frequency (e.g., more than about 100 kHz), medium frequency (e.g., between 1 kHz and 100 kHz) and low frequency (e.g., less than 1 kHz) components. As discussed hereinabove, low pass and high pass filters 1308 and 1310 may be used for the low and high components of the cumulative current, however, in certain embodiments, a function for medium frequencies may not be performed separately. In these embodiments, the nature of the high pass and low pass filters 1308 and 1310 may inherently take into consideration the medium frequency components of the unbalanced cumulative current. The summed voltage signal may be wave rectified using a wave rectifier 1314. In one embodiment, the summed voltage signal may be full wave rectified using a full wave rectifier. The full-wave rectified signal may be compared to a single analog threshold using a threshold comparator 1316. Further, in instances where the rectified signal exceeds an analog threshold, an indicator may be generated by a threshold comparator 1316 to indicate an overcurrent fault condition due to the value of the unbalanced cumulative current being higher than the threshold value for the unbalanced cumulative current. Additionally, the overcurrent fault condition may be processed with a glitch removal circuit 1318 to identify and alleviate latching false overcurrent faults caused by insignificant short-duration interferences captured by the reference electrode 1302. In an example embodiment, a trip may be initiated upon identification of the overcurrent fault condition. In certain embodiments, the term “trip” may be used to refer to an event that occurs to operatively disconnect the plurality of electrical channels from the patient. In one example, the trip condition may be prompted by the overcurrent fault condition. Initiating the trip by the glitch removal circuit 1318 results in operative decoupling of all electrical channels from the patient while the electrodes are still physically coupled to the patient. Further, it may be noted that in some embodiments, the glitch removal circuit 1318 may be optional.

It may be noted that the low pass filter components, high pass filter components, weighted combined components, wave rectifier, and threshold comparator may be selected such that the overall frequency response and threshold at which a trip occurs approximates the maximum current profile of the IEC 60601-1 standards. The overall frequency response of the reference current monitor 1304 provides more attenuation or less amplification for high frequency signals, thus allowing more high frequency currents before exceeding the single analog threshold. Further, the overall frequency response provides less attenuation or more amplification for low frequency signals, thus allowing less low frequency currents before exceeding the single-analog threshold. The overlap in response of the high pass and low pass filters passes medium frequency with a proportional attenuation, thus allowing medium frequency currents to approximate the IEC 60601-1 threshold before exceeding the single analog threshold.

In one embodiment, although not illustrated, the output of the reference current monitor, HWFAULT 1320 may be configured to drive a logic circuitry. In one example, the logic circuitry may be a digital logic circuitry. The digital logic circuitry driven by the HWFAULT 1320 may be used to automatically operatively disconnect the channels from the plurality of electrodes. By way of example, the channels may be disconnected from the plurality of electrodes by turning off series switches or relays in each channel. In embodiments of the present specification, the hardware digital logic is configured to perform functionality that may otherwise generally be performed using software.

It may be noted that similar to the regular Howland circuit, a wideband implementation in the reference current monitor may indicate that the trip limit needs to be set at a low value to be below the threshold value defined by the IEC 60601-1 current limits for low frequencies. Further, a low current limit over a frequency spectrum may have drawbacks. In one example, the low current limit may result in a greater probability for false trips if the cumulative current at the reference electrode is not balanced. In another example, when the cumulative current is purposely unbalanced, the trip may occur sooner than is desired in instances where the low current limit over the frequency spectrum is sufficiently below the threshold value defined by the IEC 60601-1 current limits.

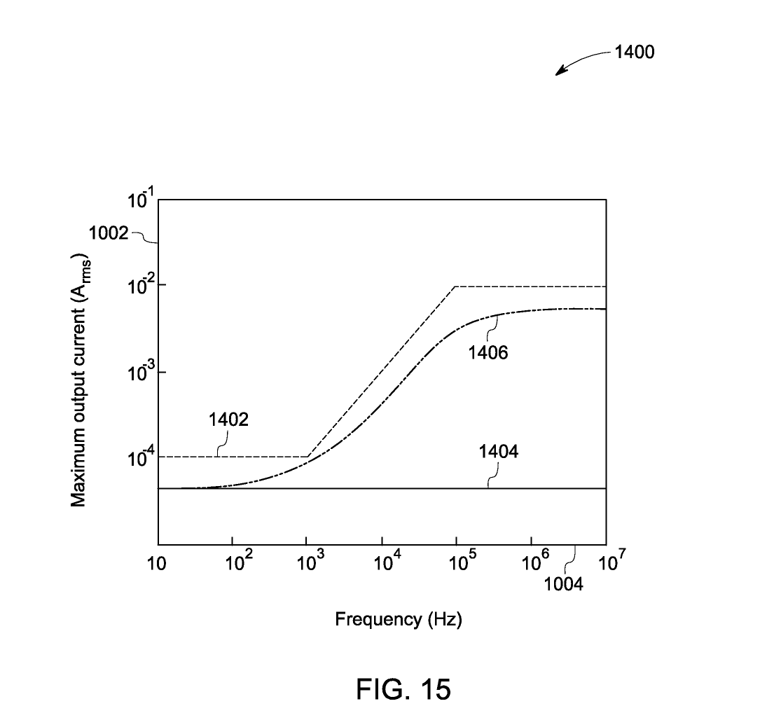

FIG. 15 illustrates simulation results 1400 for a reference current monitor (not shown) configured to detect an overcurrent fault condition at the reference electrode. In one example, one or more error conditions in the system may result in undesirably high cumulative current at the reference electrode. A non-limiting example of the error conditions may include a condition where an electrode is not properly coupled to the patient, thus unintentionally introducing an unexpected current imbalance. In instances where the overcurrent fault condition exists or is detected, the monitoring and control unit may be configured to shut down all channels of the plurality of channels that are coupled to the respective electrode current sources, wherein the electrode current sources in turn are coupled to the corresponding electrodes. Advantageously, the reference current monitor trip limits, which employs a weighed sum of low pass filter and high pass filter components of the wideband signal, are shaped into an S-curve that tracks the IEC 60601-1 limits and mitigates the drawbacks of the wideband approach. Curve 1402 represents the IEC 60601-1 current limits, curve 1404 represents the current limits over the frequency spectrum for a wideband approach which applied low current limits for the frequency spectrum, and curve 1406 represents current limits used by the reference current monitor based on the frequency of the current. As illustrated, advantageously, the curve 1406 represents higher current limits while still following the IEC 60601-1 current limits

In certain embodiments, an electrical impedance imaging system employs two redundant reference current monitors connected in parallel to a reference electrode. In these embodiments, each of the reference current monitors may be connected with a series resistor to a virtual reference. Accordingly, the current into the reference electrode may split evenly between the two reference current monitors arranged in parallel combination with respect to each other. Advantageously, the redundant reference current monitors may be implemented so that a failure in one reference current monitor may be tolerated without loss of patient protection.

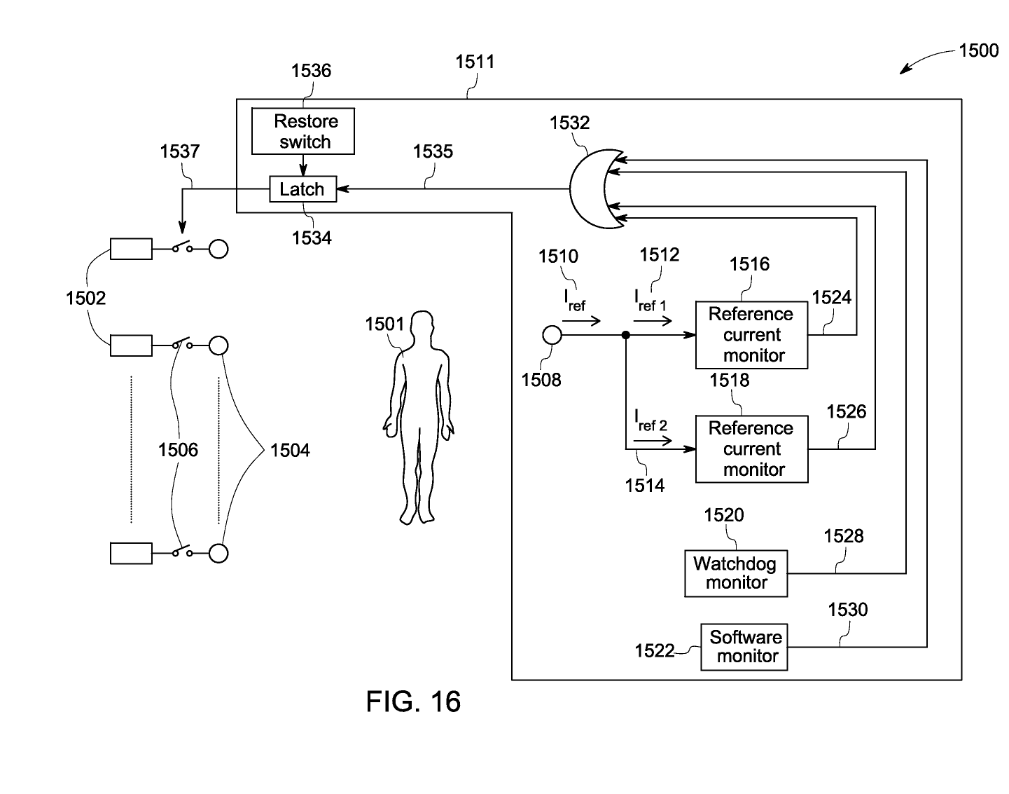

FIG. 16 illustrates a portion 1500 of an electrical impedance imaging system (not shown) configured to provide electrical impedance imaging of a subject 1501. The portion 1500 includes a plurality of electrical channels 1502, a plurality of electrodes 1504 and a reference electrode 1508. The portion 1500 further includes a monitoring and control unit 1511 configured to monitor the cumulative current at the reference electrode 1508. Further, the monitoring and control unit 1511 is configured to provide suitable action if the overcurrent fault condition is detected at the reference electrode 1508. In the illustrated embodiment, the plurality of electrical channels 1502 may have inbuilt current limiting.

With inbuilt current limiting in each electrical channel 1502, the individual electrical channels are safe, however, in instances where the sum of the currents from the plurality of electrical channels 1502 may exceed the reference threshold value for the cumulative currents, the monitoring and control unit 1511 may be used to take suitable action to provide protection to the subject 1501. In certain embodiments, the monitoring and control unit 1511 may be configured to electrically disconnect the electrical channels 1502 in the event of an overcurrent fault condition at the reference electrode 1508. Further, the monitoring and control unit 1511 may be configured to electrically disconnect the electrical channels 1502 in the event of a software related fault identified by a software monitor 1522 or a watchdog related fault identified by a watchdog monitor 1520. In one embodiment, the software monitor may be a part of a processor unit. To permit single-fault tolerance, elements of the monitoring and control unit 1511 may be implemented with dual redundancy. The reference electrode 1508 is coupled to two reference current monitors 1516 and 1518. Further, the reference current monitors 1516 and 1518 are connected in parallel. In the non-limiting illustrated example of FIG. 16, the current (I_{ref}) 1510 collected at the reference electrode 1508 may be divided between the reference current monitors 1516 and 1518. The current 1510 may be divided evenly or unevenly between the reference current monitors 1516 and 1518. The currents to the reference current monitors 1516 and 1518 may be represented as I_{ref1} 1512 and I_{ref2} 1514, respectively. If one or both of the reference current monitors 1516 and 1518 indicate an overcurrent fault condition, the monitoring and control unit 1511 indicates the fault in hardware as hardware faults 1526 and/or 1528. Once faults are detected from software fault 1530, watchdog fault 1528, and/or hardware faults 1526 and 1528, the monitoring and control unit 1511 operatively disconnects the electrical channels 1502 from their respective electrodes 1504. In one embodiment, each electrical channel 1502 may include an electrically controlled switch 1506. Each switch 1506 of a plurality of switches 1506 may be configured to electrically connect and disconnect the corresponding electrical channels 1502 from their respective electrodes 1504. The switches 1506 may be operatively coupled to the monitoring and control unit 1511. Further, the switches 1506 may be controlled using the monitoring and control unit 1511. For example, upon detection of a fault condition, the switches 1506 may be switched off based on a signal 1537 received from the monitoring and control unit 1511 to operatively disconnect the individual electrical channels 1502 from their respective electrodes 1504. Once the faults are cleared, connections, such as, but not limited to, connections between the electrical channels 1502 and the electrodes 1504 may be restored. In one example, a restore switch 1536 may be manually pressed to restore the system connection.

In certain embodiments, a fault signal may be generated based on outputs from one or more monitors 1516, 1518, 1520 and 1522, respectively. By way of example, if the output of the watchdog monitor 1520 indicates that a watchdog fault has occurred, however, the output of the software monitor 1522 does not indicate any fault, the fault signal may be generated based on the output of the watchdog monitor 1520. In addition to the intrinsic hardware fault detecting system, a latch 1534 may be employed by the system.

In the event the software monitor 1522 fails to recognize an operating fault condition, the watchdog monitor 1520 may provide an indicator of abnormal operation. In certain embodiments, the system software produces a watchdog pulse signal on a periodic basis. In certain embodiments, the watchdog pulse signal is configured to monitor the system software. In one example, a watchdog pulse signal may be transmitted at the beginning of iteration of each loop of the one or more loops that the software executes at regular intervals.

In the case of a watchdog fault condition where the watchdog pulses are no longer present at the expected rate, it is determined that a problem may have occurred with the software that prevented the loop from completing (i.e. the software has hung/stalled). Further, in one embodiment, the watchdog monitor may be configured to monitor the periodicity of a status signal that is output from the processor unit. In the event that the software has a running fault, the periodicity of the watchdog signal may change. In such instances of discrepancies in the periodicity of the watchdog signal, the watchdog monitor 1520 may provide an output signal that is indicative of a fault. For example, if the watchdog signal arrives too early or if the watchdog signal arrives too late or fails to arrive, the watchdog output signal may indicate a fault.

In some embodiments, the software and watchdog output signals may be connected to a logic block 1532 (e.g., a logical OR function) along with the output signals of the reference current monitors 1516 and 1518. Further, the output signal lines 1524, 1526, 1528 and 1530 from the monitors 1516, 1518, 1520 and 1522, respectively, may be coupled to the hardware configured to control the connection and disconnection of the electrical channels 1502 and the electrodes 1504.

In the illustrated embodiment, the latch 1534 and the restore switch 1536 may be a part of the monitoring and control unit 1511. The latch 1534 may be set by the system software detecting an operational fault and/or a failure to receive a watchdog signal from the system software. In one embodiment, the latch may be set by any fault, including the hardware fault (e.g., fault detected by the reference current monitors 1516 and 1518).

The present specification provides hardware based monitoring and control unit for the system which does not depend on software for safe operation. There are instances where an overcurrent condition may be transient in nature such that hardware fault detected by monitors 1516 and 1518, watchdog fault, or software fault may be active for a period of time before the fault disappears. When any of these hardware, watchdog or software faults initially occurs, the latch is used to store the value of “FAULT”, generally represented by reference numeral 1535, as a determined value, so that in the event that the fault condition is cleared, the value “FAULT” 1535 may remain active. When the restore switch 1536 is activated, then the determined value of “FAULT” 1535 may be reset to allow the electrical channels 1502 to be operatively connected to the respective electrodes 1504.

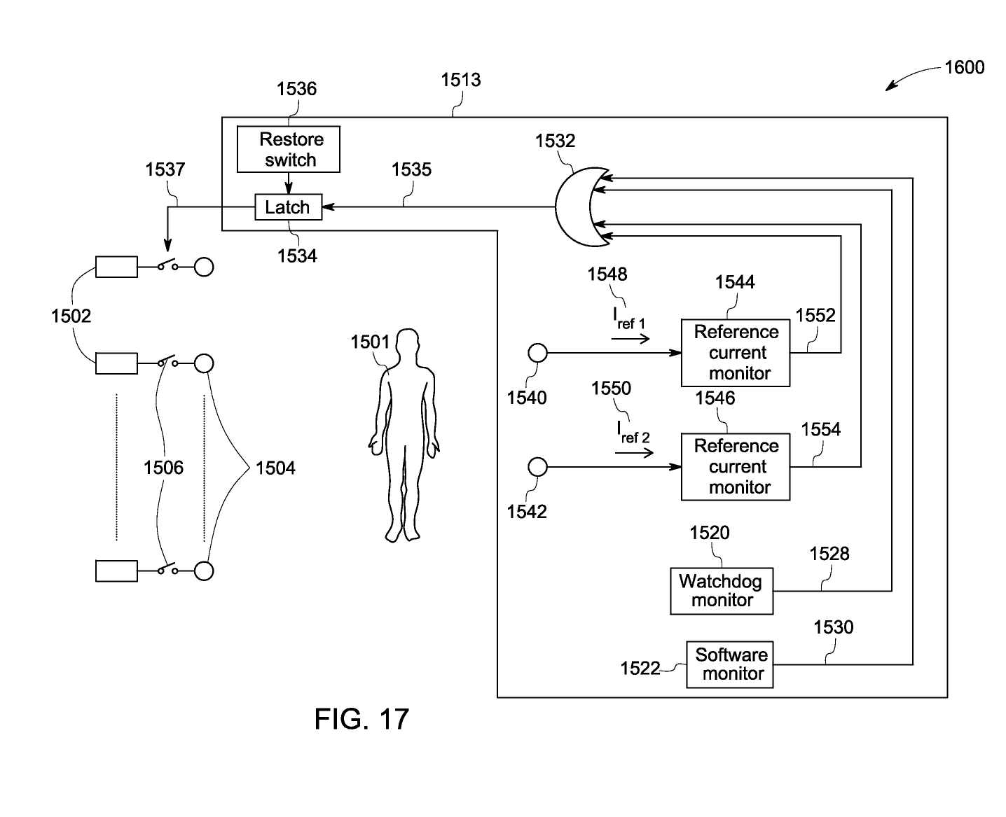

FIG. 17 illustrates an alternative embodiment of the portion 1500 of an electrical impedance imaging system. In the illustrated embodiment, the portion 1600 of an electrical impedance imaging system may include a monitoring and control unit 1513 that employs two reference electrodes 1540 and 1542. Further, the reference electrode 1540 may be coupled to a reference current monitor 1544 having an output line 1552. Also, the reference electrode 1542 may be coupled to a reference current monitor 1546 having an output line 1554. Although not illustrated, the reference electrodes 1540 and 1542 may be coupled in parallel. Further, the reference electrodes 1540 and 1542 may be coupled to a common reference electrode. Additionally, the common reference electrode may be configured to receive cumulative currents from the plurality of electrodes 1504. The cumulative current may be divided between the reference electrodes 1540 and 1542. In the illustrated embodiment, the reference electrode 1540 may receive the reference current, I_{ref1} 1548, and the reference electrode 1542 may receive the reference current, I_{ref2} 1550. Further, although not illustrated, in some embodiments, the reference electrodes 1540 and 1542 may be coupled to two or more reference current monitors.

It should be noted that there may be some safety-related software failures. For example, a first safety-related software failure may be when the software incorrectly computes and/or commands the hardware to output undesired currents, potentially greater than the regulatory limits A second safety-related software failure may be when the software enters a known error condition that is detected through error handling code (i.e. power supply voltages or currents out of range, sampled voltages too large from nodes within the reference current monitors). A third safety-related software failure may be when the software operation becomes non-deterministic (i.e. stuck in a loop, unanticipated logic state, etc.).

The first safety-related software failure may be controlled by the incorporation of current limiting within each channel as well as the measurement of cumulative current to the patient through the monitoring and control unit. In particular, the first safety-related software failure may be controlled by the use of the output signals from the reference current monitor. The second safety-related software failure may be detected within the software through error handling code and appropriate action can be taken to operatively disconnect the outputs using the software fault. The third safety-related software failure may be addressed by using a watchdog timer circuit to generate the watchdog fault signal as part of the monitoring and control unit.

In certain embodiments, the safety approach of the present specification may be integrated into the electrical channels and electrode current source for electrical impedance imaging. Advantageously, the electrode current source is designed to be a high output impedance current source. In certain embodiments, the high output impedance current source may be provided with the combined use of a current source circuit and negative impedance converter. The current source, circuit boards, and patient electrodes may have stray impedances which normally reduces the effective output impedance of a current source. By using the negative impedance converter, this stray impedance may be cancelled, which allows the full circuit to attain high output impedance. The use of inbuilt frequency-sensitive passive current limiting of the present application is applicable to both voltage and current sources.

Advantageously, the monitoring and control unit and electrode current sources provide an electrical impedance imaging system with high output impedance current sources with inherent patient protection over the entire frequency spectrum. Additionally, parasitic impedance in a high output impedance current source is removed using a negative-impedance converter. The use of negative impedance converter is associated with instability in the circuit and this instability in the circuit is further deteriorated due to addition of passive current limiting components. This challenge of accomplishing circuit stability while using a negative impedance converter is achieved by careful compensation of the circuit in a non-obvious fashion. As discussed above, the hardware-based safety approaches are preferred over software-based approaches by the regulatory bodies.

Moreover, the embodiments pertaining to the electrode current source of the present specification are configured to provide at least the advantages of (1) protection over the entire frequency spectrum to track the IEC 60601-1 limits, (2) high output impedance and circuit stability, (3) high SNR due to the ability to output more current than otherwise possible in a wideband approach, and (4) the use of only hardware circuits to provide current limiting without using any software. Additionally, the embodiments pertaining to the monitoring and control unit provide the advantages of (1) protection over the entire frequency spectrum to track the current limits defined by the regulatory bodies, (2) reduction of false trips and the ability to purposely unbalance more than a conventional wideband approach, and (3) the use of only hardware circuits without the need for any software.

While only certain features of the present specification have been illustrated and described herein, many modifications and changes will occur to those skilled in the art. It is, therefore, to be understood that the appended claims are intended to cover all such modifications and changes as fall within the scope of the present specification.

1. An electrical channel having inbuilt current limiting, comprising:
an electrode current source, comprising:
a voltage-to-current converter configured to receive an input voltage and output a corresponding output current;
a negative impedance converter operatively coupled to the voltage-to-current converter, wherein the negative impedance converter is configured to cancel an output impedance of the voltage-to-current converter, a parasitic impedance, or both; and
at least one passive current limiting component configured to limit the output current to a load below a threshold value.

2. The electrical channel of claim 1, wherein the at least one passive current limiting component is an integrated passive current limiting component.

3. The electrical channel of claim 2, wherein the integrated passive current limiting component is integrated with a circuit topology of the voltage-to-current converter, the negative impedance converter, or both.

4. The electrical channel of claim 1, wherein the passive current limiting component is disposed between the voltage-to-current converter and the negative impedance converter, in series with the load, or both.

5. The electrical channel of claim 1, wherein the passive current limiting component comprises a first resistor operatively coupled in series to a parallel combination of a second resistor and a first capacitor.

6. The electrical channel of claim 5, further comprising a second capacitor operatively coupled to the parallel combination of the second resistor and the first capacitor.

7. The electrical channel of claim 1, wherein the passive current limiting component comprises a resistor and a capacitor in a series combination.

8. The electrical channel of claim 1, wherein the voltage-to-current converter is a Howland circuit.

9. The electrical channel of claim 1, wherein the voltage-to-current converter is an enhanced Howland circuit, and wherein the enhanced Howland circuit comprises one or more passive current limiting components, one or more passive stability components, or both.

10. The electrical channel of claim 9, wherein the enhanced Howland circuit comprises a first integrated passive current limiting component configured for low frequency current limit shaping, a second integrated passive stability component configured for high frequency circuit stability, or both.

11. The electrical channel of claim 1, wherein the negative impedance converter is an enhanced negative impedance converter, and wherein the enhanced negative impedance converter comprises one or more passive current limiting components.

12. The electrical channel of claim 11, wherein the enhanced negative impedance converter comprises a first integrated passive current limiting component configured for high frequency current limit shaping, and a second integrated passive stability component configured for high frequency circuit stability, or both.

13. The electrical channel of claim 1, further comprising an excitation source, a response detector, or both.

14. The electrical channel of claim 13, wherein the electrode current source is a part of the excitation source.

15. A reference current monitor configured to monitor a current at a reference electrode, comprising:
a reference current-to-voltage converter;
a low pass filter operatively coupled to the reference current-to-voltage converter;
a high pass filter operatively coupled to the reference current-to-voltage converter; and
a summator operatively coupled to the low and high pass filters.

16. The reference current monitor of claim 15, further comprising:
a wave rectifier operatively coupled to the summator to provide a wave rectified signal; and
a threshold comparator operatively coupled to the wave rectifier, wherein the threshold comparator is configured to compare the wave rectified signal to a threshold signal.

17. A monitoring and control unit, comprising:
one or more reference current monitors configured to monitor at least a portion of a reference current appearing at a reference electrode, wherein the one or more reference current monitors are configured to provide respective monitor output signals, and wherein each of the one or more reference current monitors comprises:
a reference current-to-voltage converter;
a low pass filter operatively coupled to the reference current-to-voltage converter;
a high pass filter operatively coupled to the reference current-to-voltage converter; and
a summator operatively coupled to the low and high pass filters.

18. The monitoring and control unit of claim 17, wherein the one or more reference current monitors further comprise a wave rectifier, a threshold comparator, or both.

19. The monitoring and control unit of claim 17, further comprising a watchdog monitor configured to detect a watchdog fault, wherein the watchdog monitor is configured to provide a watchdog output signal.

20. The monitoring and control unit of claim 19, further comprising a software monitor configured to detect a software fault, wherein the software monitor is configured to provide a software output signal.

21. The monitoring and control unit of claim 20, further comprising logic circuitry configured to receive one or more of the reference current monitor output signals, the watchdog output signal, and the software output signal to determine an existence of a fault condition.

22. The monitoring and control unit of claim 21, further comprising a latch configured to detect a hardware fault, a software fault, a watchdog fault, or combinations thereof.

23. An electrical impedance imaging system for imaging a subject, comprising:
a plurality of electrodes configured to be disposed on the subject;
a reference electrode configured to be disposed on the subject; and
a plurality of electrical channels, wherein each electrical channel of the plurality of channels is configured to be operatively coupled to a corresponding electrode of the plurality of electrodes, wherein each electrical channel of the plurality of electrical channels comprises inbuilt current limiting, and wherein each electrical channel of the plurality of electrical channels comprises:
an electrode current source, comprising:
a voltage-to-current converter configured to receive an input voltage and output a corresponding output current;
a negative impedance converter operatively coupled to the voltage-to-current converter, wherein the negative impedance converter is configured to cancel an output impedance of the voltage-to-current converter, a parasitic impedance, or both; and
at least one passive current limiting component operatively coupled to the voltage-to-current converter, the negative impedance converter, a load, or combinations thereof, and wherein the at least one passive current limiting component is configured to limit the output current to the load below a threshold value; and
a processor unit comprising a physiological parameter extraction module.

24. The electrical impedance imaging system of claim 23, further comprising a monitoring and control unit, comprising:
one or more reference current monitors operatively coupled to the reference electrode, wherein the one or more reference current monitors are configured to determine an overcurrent fault condition at the reference electrode.

25. The electrical impedance imaging system of claim 24, wherein each electrical channel of the plurality of electrical channels comprises an electrically controlled switch, wherein the electrically controlled switch is configured to selectively connect or disconnect a corresponding electrical channel and a respective electrode of the plurality of electrodes, and wherein the electrically controlled switch of each electrical channel is configured to receive an output from the monitoring and control unit.

26. The electrical impedance imaging system of claim 23, wherein the reference electrode is operatively coupled to two or more reference current monitors, and wherein the two or more reference current monitors are coupled in parallel to one another.

27. The electrical impedance imaging system of claim 23, comprising two or more reference electrodes, wherein the two or more reference electrodes are each coupled to a corresponding reference current monitor.

28. The electrical impedance imaging system of claim 23, further comprising a restore switch configured to restore system connections.

29. The electrical impedance imaging system of claim 23, further comprising one or more reference current monitors, a watchdog monitor, a software monitor, and wherein output lines from the one or more reference current monitors, the watchdog monitor, and the software monitor are coupled to a logic circuitry.
